(19) United States
(12) Patent Application Publication
(10) Pub. No.: US 2007/0009465 A1
(43) Pub. Date: Jan. 11, 2007

(54) Cosmetic compositions comprising polyol/polyester block polymers

(75) Inventors: Andreas Lendlein, Berlin (DE); Tareq Ali Abdulla Hasson, Aachen (DE); Karl Kratz, Berlin (DE); Simone Moersheim, Eschweiler (DE); Yasmin Ghazlan, Bremen-Vahr (DE); Susanne Birkel, Darmstadt (DE); Thomas Krause, Darmstadt (DE); Axel Kalbfleisch, Darmstadt (DE); Andrea Walter, Buettelborn (DE); Susanne Granner, Buettelborn (DE)

Correspondence Address: SEED INTELLECTUAL PROPERTY LAW GROUP PLLC, 701 FIFTH AVE, SUITE 5400, SEATTLE, WA, 98104, US

(73) Assignee: MNEMOSCIENCE GMBH, UEBACH-PALENBERG (DE)

(21) Appl. No.: 10/566,231

(22) PCT Filed: Jul. 14, 2004
(86) PCT No.: PCT/EP04/07799

§ 371 Date: Sep. 19, 2006

(30) Foreign Application Priority Data
Jul. 30, 2003 (DE) 103 34 784.4

(51) Int. Cl. A61K 8/84

(52) U.S. Cl. 424/70.11; 424/70.7

(57) ABSTRACT

A cosmetic composition is described, said composition having a content of a block polymer having at least a first block, which block is a polyol selected from polyethers, dimerdiol, derivatives derived from dimerdiol and polyesters of dicarboxylic acids with diols and at least two additional blocks, which blocks are polyesters of hydroxycarboxylic acids or lactones thereof.

The object of the invention under consideration is a cosmetic composition with a content of specific block polymers comprised of polyol blocks and polyester blocks, with a further object being methods for hair treatment using the block polymers.

In the shaping of hair, a distinction is generally made between temporary and lasting, permanent hair styling. As a rule, a temporary hair styling takes place with the use of compositions based on solutions or dispersions of hair-fixing polymers. Products of this kind give the hair more or less hold, volume, elasticity, springiness and lustre by means of the addition of polymers. For example, in the form of a gel, these styling products facilitate the shaping and creation of a hairstyle; in the form of a hairspray, they improve the condition of a created hairstyle, and, in the form of a fixing foam, they increase the volume of the hair. Detrimental is that the desired effects last for only a relatively short time and are quickly lost again as the result of outside influences, such as combing, wind, high air humidity, contact with water or sleeping on the hairstyle. As a rule, such influences necessitate a renewed, time-consuming hairstyle creation and a renewed application of a styling preparation. As a rule, a permanent hair styling is accomplished by means of a permanent wave treatment. In this case, disulfide bonds in the hair are reductively cleaved, the hair is brought into a new shape and this new shape is fixed by forming new disulfide bonds by means of an oxidative process. Detrimental is that, as a result of the necessary chemical treatment of the hair with reductive and oxidation agents, it is not possible to avoid damage to the hair structure. A further disadvantage of the methods for hair restyling known up to now is that it is not possible to make the restyling reversible in a relatively simple manner, i.e., to move from one hairstyle to another without complex re-creation.

The subject matter underlying the invention under consideration was to provide a composition which, after the creation of a first hairstyle, facilitates a simple, uncomplicated and quick restyling or recovery of the hairstyle, without it being absolutely necessary to re-apply the composition during the restyling. A further subject matter was to provide a method enabling retrievable hair restyling without damaging intervention in the hair structure, in particular, a method that makes it easier to prepare a hairstyle again after deformations of an originally prepared hairstyle that have been introduced by external influences.

The object of the invention is a cosmetic composition containing, in a suitable cosmetic foundation, at least one block polymer having at least a first block, which is a polyol selected from polyethers, oligoethers, hydrocarbons having a molecular weight of at least 400 g/mol and at least two alcoholic hydroxyl groups, oligoester diols and polyesters of dicarboxylic acids with diols and at least two additional blocks, which are polyesters of hydroxycarboxylic acids or their lactones. In the context of the invention, a polyol is a compound having at least two alcoholic hydroxyl groups. The hydrocarbons can be linear, branched, saturated, monounsaturated or polyunsaturated. Polyethers and oligoethers are polymer or oligomer compounds whose organic repeating units are held together by ether functionalities (C—O—C). In the context of the invention, polyethers have at least 4 ether groups and at least 5 repeating units, and oligoethers have one, two or three ether groups and from 2 to 4 repeating units.

Block polymers that are suitable according to the invention preferably consist of at least one hard segment and at least one soft segment, and preferably have at least two transition temperatures Tₜᵣₐₙₛ and T′ₜᵣₐₙₛ, wherein T′ₜᵣₐₙₛ lies above room temperature (25° C.) and Tₜᵣₐₙₛ lies below T′ₜᵣₐₙₛ.

The polymer segments are preferably oligomers or dihydroxy hydrocarbons, in particular, linear chain molecules having a molecular weight of, for example, 400 to 30,000, preferably 1,000 to 20,000 or 1,500 to 15,000. The molecular weight of the block polymers can, for example, be from 30,000 to 1,000,000, preferably from 50,000 to 700,000 or from 70,000 to 400,000. These have a degree of crystallinity of preferably from 3 to 80%, particularly preferably from 3 to 60%. The two transition temperatures can, for example, be melting temperatures Tₘ or glass transition temperatures T_{g}. Above Tₜᵣₐₙₛ, the polymer has a lower modulus of elasticity than it does below Tₜᵣₐₙₛ. The ratio of the moduli of elasticity below and above Tₜᵣₐₙₛ as is preferably at least 10, particularly preferably at least 20. The lower transition temperature Tₜᵣₐₙₛ is preferably greater than room temperature (25° C.), in particular at least 30° C., particularly preferably at least 35° C. or at least 40° C., and is that temperature which, when exceeded, sees the spontaneous recovery of the permanent shape from the deformed shape or from the temporary shape. Tₜᵣₐₙₛ is preferably so far above the usually to be expected ambient temperatures that at the ambient temperature, no significant, unintentional, thermally induced re-shaping of the temporary hairstyle occurs. Suitable ranges for Tₜᵣₐₙₛ are, for example, from 25 to 100° C., from 30 to 75° C., from 35 to 70° C. or from 40 to 60° C. The upper transition temperature T′ₜᵣₐₙₛ is greater than Tₜᵣₐₙₛ and is that temperature above which the impression of the permanent shape or the re-impression of a permanent shape into a new permanent shape occurs, and below which the permanent shape is fixed. T′ₜᵣₐₙₛ is preferably so much greater than Tₜᵣₐₙₛ that during the heating of the hairstyle to a temperature above Tₜᵣₐₙₛ for the recovery of the permanent hairstyle or for the reforming of a temporary hairstyle while maintaining the permanent hairstyle, no significant, unintentional, thermally induced deformation of the permanent shape of the hairstyle occurs. Preferably, T′ₜᵣₐₙₛ is at least 10° C., particularly preferably at least 20° C. or at least 30° C. above Tₜᵣₐₙₛ. The difference between T′ₜᵣₐₙₛ and Tₜᵣₐₙₛ can, for example, be from 10 to 80° C., from 20 to 70° C. or from 30 to 60° C. Suitable ranges for T′ₜᵣₐₙₛ are, for example, from 40 to 150° C., from 50 to 100° C. or from 70 to 95° C.

Suitable polymers P are, for example, polymers having the following general formula
A(B)ₙ (I)
wherein A is derived from an n-valent polyether or oligoether, from hydrocarbons having a molecular weight of at least 400 g/mol and n alcoholic hydroxyl groups, from oligoester diols or from a polyester of a dicarboxylic acid with one diol, B represents a poly(hydroxycarboxylic acid) block and n represents a number greater than or equal to two, preferably 2, 3 or 4. Preferred are B-A-B triblock polymers, particularly block polymers with a single polyol block as the middle block, two terminal polyester blocks of hydroxycarboxylic acids or their lactones and terminal alcoholic hydroxyl groups.

A preferably represents polyalkylene glycol ethers from polyvalent alcohols, poly(tetrahydrofurane), dimerdiol, dimerdiol oligoethers and oligoester diols (dihydroxy oligoesters). The polyalkylene glycol ethers preferably have 2 to 6 C atoms per alkylene group; particularly preferred are poly(ethylene glycol) (PEG) and poly(propylene glycol) (PPG). Dimerdiol is the designation for alpha, omega C36 diols, which can be produced by the dimerization of oleyl alcohol or by the hydrogenation of dimer fatty acids. Dimer fatty acid is a mixture of aliphatic, branched or cyclic C36 dicarboxylic acids (dimer acid), which can be produced by the dimerization of oleic acid or tall oil fatty acid (TOFA). Dimerdiol can be obtained, for example, under the trade name Sovermol® 908. Dimerdiol oligoethers are oligomers of dimerdiol, and can be produced by acid-catalyzed dehydration of dimerdiol. Preferred are dimers, trimers and tetramers of dimerdiol. Suitable commercial products are, e.g., Sovermol® 909 with a molecular weight of approximately 1,000 or Sovermol® 910 with a molecular weight of approximately 2,000. Oligoester diols (dihydroxy oligoesters) are reaction products of an oligomerization of a dicarboxylic acid with a diol, wherein the reaction product has two hydroxyl groups. Suitable dicarboxylic acids are, for example, C3 to C20 dicarboxylic acids, preferably aliphatic C4 to C10 dicarboxylic acids. Aliphatic dicarboxylic acids are, for example, oxalic acid, malonic acid, diacetic acid, glutaric acid, adipic acid, pimelic acid, azelaic acid, sebacic acid, maleic acid and fumaric acid. Aromatic dicarboxylic acids are, for example, phthalic acid and terephthalic acid. Diol components of the oligoester diols are, for example, C2 to C30 diols, preferably aliphatic C5 to C20 diols. Also suitable are glycerol monoesters, particularly monoesters of glycerol with aliphatic C2 to C30 monocarboxylic acids, preferably C5 to C20 monocarboxylic acids.

The poly(hydroxycarboxylic acid) block B can be formed of hydroxycarboxylic acids, particularly monohydroxymonocarboxylic acids having up to 30 C atoms, as well as of their lactones or lactides. The poly(hydroxycarboxylic acid) block B can also be a copolymer of two or more different hydroxycarboxylic acids. Hydroxycarboxylic acids can be: saturated or unsaturated aliphatic hydroxycarboxylic acids, aromatic hydroxycarboxylic acids, alpha-hydroxycarboxylic acids, beta-hydroxycarboxylic acids, omega-hydroxycarboxylic acids and hydroxy fatty acids. Alpha-hydroxycarboxylic acids are, for example, glycolic acid, lactic acid or mandelic acid. Beta-hydroxycarboxylic acids are, for example, beta hydroxyalkyl acids such as beta hydroxybutyric acid or beta-hydroxyvaleric acid. Hydroxy fatty acids are, for example, 12-hydroxystearic acid or ricinoleic acid. Aromatic hydroxy acids are, for example, hydroxybenzoic acids, e.g., salicylic acid.

B preferably stands for poly(ε-caprolactone), polylactides, polyglycolides, poly(lactide-co-glycolide), poly(pentadecalactone), poly(caprolactone-co-lactide), poly(pentadecalactone-co-lactide) or poly(pentadecalactone-co-caprolactone). A polyester that is particularly preferred for use according to the invention is a polyester on the basis of lactide units or pentadecalactone units.

Preferred block copolymers are those with the general formula:
HO—[B1-C(═O)O—]ₙ₁[Y—O—]ₙ₂[C(═O)—B2-O—]ₙ₃H (Ia)
B1 and B2 are the same or different and stand for branched, cyclic or linear alkylene groups with 1 to 40, preferably 2 to 20, C atoms. Y stands for a branched, cyclic or linear alkylene group with 2 to 30 C atoms, preferably for ethylene groups and/or propylene groups, or for a block comprising dimerdiol, dimerdiol oligoether or oligoester diol, wherein n1, n2 and n3 are the same or different numbers greater than or equal to zero, wherein both n2 and the sum n1+n2 are greater than zero.

Particularly preferred are polymers with the formula (Ia), wherein B1 and B2 stand for branched or linear alkylene groups with 2 to 20 C atoms, Y is an ethylene group and n1, n2 and n3 are greater than zero and are selected in such a way that the molecular weight of the polymer is greater than or equal to 2,000. Some examples of suitable polymers are block copolymers with a polyethylene glycol middle block and terminal blocks of polylactic acid or poly-ε-caprolactone, wherein the terminal blocks are end-substituted with hydroxyl groups, the middle block has a molecular weight in the range from 500 to 20,000, preferably 2,000 to 10,000, and the average molecular weight of the block copolymer is from 2,000 to 50,000, preferably from 3,000 to 25,000.

The advantage of the copolymers with a block structure is that the various properties and functions of the blocks can be combined in one polymer. The properties of the blocks, such as the hydrophilicity, the affinity to hair or the transition temperatures, can be selected independently of one another.

For example, to produce the block copolymers, an oligomeric or polymeric diol can be used as a di-functional initiator for a ring-opening polymerization (ROP). In this case, the initiator represents the A block. Preferably, polyether diols, which are commercially available in various molecular weights, are used as initiators. Preferred for use are PEO or PEG with a molecular weight of from 4,000 to 8,000 g/mol, particularly preferably with a molecular weight of 6,000 and 8,000 g/mol, which corresponds to the A block length.

Additional preferred diols are:

Dimerdiol (dimer fatty diol), commercially available under the trade name Sovermol® 908, with a molecular weight of 550 g/mol. The production of dimerdiol is known. For example, dimerdiol can be produced by the hydrogenation of dimeric oleic acid and/or its esters according to the German printed patent specification DE-B 17 68 313. The general formula is HO—CH₂—C₃₄H₆₆—CH₂—OH.

Dimerdiol oligoether, commercially available under the trade name Sovermol® 909, with a molecular weight of approximately 1,000 g/mol, and Sovermol® 910, with a molecular weight of approximately 2,000 g/mol. The general formula is HO—CH₂—C₃₄H₆₆—CH₂—O—[CH₂—C₃₄H₆₆—CH₂—O—]ₙH wherein n is a number from 1 to 5, preferably 1, 2 or 3.

Oligoester diols with molecular weights between 1,000 and 6,000 g/mol, which were synthesized starting from dicarboxylic acids and diols on a fatty basis. The diols on a fatty basis are, in particular, linear or branched aliphatic C₂ to C₁₄ diols, 12-hydroxystearyl alcohol, dimer fatty diol, dimer fatty diol oligoether with a molecular weight of from 1,000 to 2,000, and glycerol monoester with oleic acid, stearic acid or lauric acid. The dicarboxylic acids are preferably dimer fatty acid, adipic acid and azelaic acid.

Oligoester diols having the following general formulas are suitable:

Oligoester diols of dimerdiol:
HO—CH₂—C₃₄H₆₆—CH₂—O—[(O═)C—R—C(═O)—O—CH₂—C₃₄H₆₆—CH₂—O—]ₙH

Oligoester diols of 12-hydroxystearyl alcohol:
HO—(CH₂)₁₁—CH(C₆H₁₃)—O—[(O═)C—R—C(═O)—O—(CH2)₁₁-CH(C₆H₁₃)—O—]ₙH
Particularly preferred are hydroxystearyl alcohol-adipate-diol and hydroxystearyl alcohol-azelate-diol.

Oligoester diols of glycerol mono stearate:
HO—CH(CH₂—O—C(═O)(C₁₇H₃₅))—CH₂O—[(O═)C—R—C(═O)—O—CH₂—CH—(CH₂O—C(═O)—(C₁₇H₃₅))—CH₂O—]ₙH
In all of these formulas, R stands for linear, aliphatic hydrocarbons with 4 to 36 C atoms and n stands for numbers greater than or equal to 1, preferably greater than or equal to 2.

The production of oligoester diols from diols and dicarboxylic acids is known in the state of the art. The production can take place with or without the use of catalysts. All esterification catalysts, such as sulphuric acid, phosphoric acid or p-toluenesulphonic acid can be used as catalysts. Tin compounds, such as tin dioctoate, tin oxide and tin oxalate, and titanium compounds, such as titanium (IV)isopropoxide, can also be considered. Preferred are p-toluenesulphonic acid and titanium(IV)isopropoxide.

To introduce the B blocks, cyclic esters or diesters can be used, for example, dilactide, diglycolide, p-dioxanone, ε-caprolactone, ω-pentadecalactone or their mixtures. Preferred for use are dilactide, L,L-dilactide or ω-pentadecalactone. Preferably, the reaction takes place in the mass, optionally with the addition of a catalyst, such as dibutyltin(IV)oxide, dibutyltin(IV)dilaurate, tin(II)dilaurate, tin(II)octanoate, titanium(IV)isopropoxide or lithium chloride. Preferred are dibutyltin(IV)oxide and tin(II)octanoate, particularly dibutyltin(IV)oxide. If a catalyst is used, then it can be in the amount of from 0.1 to 0.3 percent by mol. Because many of the possible catalysts, particularly the tin compounds, are toxic, if the triblock copolymers are used in materials for the cosmetic or medical areas, the catalyst residue that remains in the copolymer must be removed. The appropriate operational conditions are known to the expert and illustrated by the following examples.

The B block length can be variably adjusted via the molar relationship of the monomer to initiator. The percent of the weight in A blocks preferably amounts to 40 to 90% in the block copolymers.

Additional suitable block copolymers are, for example, multi-block copolymers, which can be obtained through the reaction of a diol as initiator (block A), first with a first monomer (block B), then with a second monomer (block C).

Suitable block copolymers are, for example, also star-shaped block copolymers, which can be obtained if a triol or tetrol is used as the initiator, instead of the diol. Preferred for use as tri-functional initiators are: commercially available glycerol ethoxylate or glycerol propoxylate or glycerol-propoxylate-b-ethoxylate with a molecular weight of from 200 to 6,000 g/mol, particularly preferred with a molecular weight of 1,000 and 3,000 g/mol. Preferred for use as tetra-functional initiators are: commercially available pentaerythrite-ethoxylate or pentaerythrite-propoxylate or pentaerythrite-propoxylate-b-ethoxylate with a molecular weight of from 200 to 3,000 g/mol, particularly preferred with a molecular weight of 500 and 2,000 g/mol. In principle, diblock copolymers are also suitable.

The described block polymers are preferably equipped with hydroxyl groups at the terminals.

Compositions for the treatment of hair according to the invention contain at least one of the above-mentioned block polymers in an amount of preferably 0.01 to 25 percent by weight, particularly preferred from 0.1 to 15 percent by weight, in a suitable, preferably liquid, viscous or semi-solid medium. The composition may be present as a solution, dispersion, emulsion, suspension or latex. The liquid medium is preferably cosmetically acceptable and physiologically harmless.

The composition according to the invention is generally present in the form of a solution or dispersion in a suitable solvent. Particularly preferred are aqueous, alcoholic or aqueous-alcoholic solvents. Suitable solvents are, e.g., aliphatic C1 to C4 alcohols or a mixture of water with one of these alcohols. Other organic solvents can, however, also be employed, wherein, in particular, unbranched or branched hydrocarbons such as pentane, hexane, isopentane; cyclic hydrocarbons such as cyclopentane and cyclohexane; organic linear or cyclic ethers, such as tetrahydrofurane (THF); or liquid organic esters, such as ethylacetate, can be mentioned. Furthermore, solvents on a silicone basis are also suitable, in particular, silicone oils on the basis of linear or cyclic polydimethylsiloxanes (dimethicones or cyclomethicones). The solvents are preferably present in an amount of from 0.5 to 99 percent by weight or particularly preferably in an amount of from 40 to 90 percent by weight.

The compositions according to the invention can additionally contain from 0.01 to 25 percent by weight of at least one hair-care active ingredient, hair-fixative active ingredient and/or hair-colouring active ingredient.

Hair-fixative active ingredients are, in particular, the known conventional film-forming and hair-fixative polymers. The film-forming and hair-fixative polymer can be of synthetic origin or of natural origin, and can be of nonionic, cationic, anionic or amphoteric character. Such a polymer additive, which can be present in an amount of from 0.01 to 25 percent by weight, preferably 0.1 to 20 percent by weight, particularly preferably from 0.5 to 15 percent by weight, can also consist of a mixture of more than one polymer, and this additive can be modified further with respect to the hair-fixative properties by means of the addition of further polymers having a thickening effect. Film-forming, hair-fixative polymers according to the invention are polymers which, when employed in an aqueous, alcoholic or aqueous-alcoholic solution at a concentration of from 0.01 to 5%, are able to deposit a polymer film on the hair, thereby fixing the hair in this manner.

Homopolymers of vinyl pyrrolidone, homopolymers of N-vinyl formamide, copolymers of vinyl pyrrolidone and vinyl acetate, terpolymers of vinyl pyrrolidone, vinyl acetate and vinyl propionate, polyacrylamides, polyvinyl alcohols or polyethylene glycols having a molecular weight of from 800 to 20,000 g/mol can be employed as suitable, synthetic, nonionic, film-forming, hair-fixative polymers in the hair treatment composition according to the invention. Suitable synthetic, anionic, film-forming polymers are crotonic acid/vinyl acetate copolymers and terpolymers of acrylic acid, ethyl acrylate and N-t-butylacrylamide. Natural film-forming polymers or polymers produced therefrom by means of chemical conversion can also be used in the hair treatment composition according to the invention. Low-molecular chitosan with a molecular weight of 30,000 to 70,000 g/mol or high-molecular chitosan, organo-soluble derivatives of chitosan, mixtures of oligosaccharides, monosaccharides and disaccharides, Chinese balsam resin, cellulose derivatives such as hydroxypropylcellulose having a molecular weight of from 30,000 to 50,000 g/mol, or shellac in a neutralized or unneutralized form have proven satisfactory. Amphoteric polymers can also be employed in the hair treatment composition according to the invention. Suitable are, for example, copolymers of octylacrylamide, t-butylaminoethylmethacrylate and two or more monomers selected from the group consisting of acrylic acid, methacrylic acid and simple esters thereof. Among the cationic polymers that can be used according to the invention, copolymers of vinylpyrrolidone with quaternated derivatives of dialkyl amino acrylate and dialkyl amino methacrylate, such as vinylpyrrolidone/dimethylamino methacrylate copolymers quaternated with diethyl sulphate, can be mentioned. Additional cationic polymers are, for example, the copolymer of vinylpyrrolidone with vinylimidazolium methochloride, the terpolymer from dimethyl diallyl ammonium chloride, sodium acrylate and acrylamide, the terpolymer from vinylpyrrolidone, dimethylamino ethyl methacrylate and vinyl caprolactam, the quaternated ammonium salt produced from hydroxyethylcellulose and an epoxide substituted with trimethylammonium, the vinylpyrrolidone/methacrylamidopropyltrimethyl ammonium chloride copolymer and diquaternary polydimethylsiloxanes.

The consistency of the hair treatment composition according to the invention can be increased by means of the addition of thickeners. In this respect, homopolymers of acrylic acid having a molecular weight of from 2,000,000 to 6,000,000 g/mol are suitable, for example. Copolymers of acrylic acid and acrylamide (sodium salt) having a molecular weight of from 2,000,000 to 6,000,000 g/mol, sclerotium gum and copolymers of acrylic acid and methacrylic acid are also suitable.

A cosmetic composition according to the invention can be used in various forms of application, for example, as a lotion, a spray lotion, a cream, a gel, a foam-gel, an aerosol spray, a non-aerosol spray, an aerosol foam, a non-aerosol foam, an o/w emulsion or w/o emulsion, a micro emulsion or a hair wax.

If the hair treatment composition according to the invention is provided in the form of an aerosol spray, the composition additionally contains from 15 to 85 percent by weight, preferably from 25 to 75 percent by weight, of a propellant, and the composition is filled into a pressurized container having a spray head. Suitable as propellants are lower alkanes, such as n-butane, isobutene and propane, as well as mixtures thereof, as well as dimethyl ethers or fluorohydrocarbons, such as F 152a (1,1-difluoroethane) or F 134 (tetrafluoroethane), as well as propellants which are present at the pressures in question in gaseous forms, such as N₂, N₂O and CO₂, as well as mixtures of the propellants mentioned in the preceding.

If the hair treatment composition according to the invention is provided in the form of a sprayable non-aerosol hairspray, the composition is sprayed by means of a suitable, mechanical spraying device. Spray devices of the mechanical type are devices which allow the spraying of a composition without the use of a propellant. Suitable mechanical spray devices are, e.g., spray pumps, or elastic containers provided with a spray valve, into which the cosmetic composition according to the invention is filled under pressure, wherein the elastic container is expanded and wherein the cosmetic composition is continuously discharged if the valve is open, due to the contraction of the elastic container.

If the hair treatment composition according to the invention is provided in the form of a hair foam (mousse), the composition contains at least one conventional foaming substance known in the art for this purpose. The composition is foamed with or without the aid of propellant gases or chemical propellants and worked into the hair as a foam, and then left in the hair without being rinsed out. A product according to the invention has, as an additional component, a device for foaming the composition. Considered as devices for foaming are devices which allow a liquid to be foamed, with or without the use of a propellant. For example, a commercially available foam pump or an aerosol foaming head can be used as a suitable mechanical foaming device.

If the hair treatment composition according to the invention is provided in the form of a hair gel, it additionally contains at least one gel-forming substance in an amount of preferably from 0.05 to 10, particularly preferably from 0.1 to 2, percent by weight. The viscosity of the gel preferably amounts to from 100 to 50,000 mm²/s, particularly preferably from 1,000 to 15,000 mm²/s, at 25° C., measured as dynamic viscosity using a Bohlin Rheometer CS, measurement body C25 at a shear velocity of 50 s⁻¹.

If the hair treatment composition according to the invention is provided in the form of a hair wax, it additionally contains water-insoluble fatty substances or waxy substances, or substances that provide the composition with a waxy consistency, in an amount of preferably 0.5 to 30 percent by weight. Suitable water-insoluble substances are, for example, emulsifiers having an HLB-value of below 7, silicone oils, silicone waxes, wax materials (e.g., wax alcohols, wax acids, wax esters, as well as, in particular, natural waxes such as beeswax, Carnauba wax, etc.), fatty alcohols, fatty acids, fatty acid esters or high-molecular polyethylene glycols having a molecular weight of from 800 to 20,000, preferably from 2,000 to 10,000, g/mol.

If the hair treatment composition according to the invention is provided in the form of a hair lotion, it is present as a substantially non-viscous or low-viscous solution, dispersion or emulsion, each capable of flowing, with a content of at least 10 percent by weight, preferably 20 to 95 percent by weight, of a cosmetically compatible alcohol. Suitable for use as alcohols are particularly the lower C1 to C4 alcohols usually used for cosmetic purposes, such as ethanol and isopropanol, for example.

If the hair treatment composition according to the invention is present in the form of a hair cream, it is preferably provided in the form of an emulsion and either it contains additional viscosity-providing components in an amount of from 0.1 to 10 percent by weight, or the required viscosity and creamy consistency are built up in the normal way by means of micelle formation, with the help of suitable emulsifiers, fatty acids, fatty alcohols, waxes, etc.

In a preferred embodiment, the composition according to the invention is capable of simultaneously facilitating both improved creation of the hairstyle and the provision of hair coloration. The composition is then formulated as a colouring hair treatment composition, e.g., as a colouring fixative, a colouring cream, a colouring foam, etc. It then contains at least one colouring substance. This colouring substance component can be an organic colorant, in particular a so-called direct dye, or it can also involve inorganic pigments.

The total amount of colouring substances in the composition according to the invention is roughly 0.01 to 7 percent by weight, preferably roughly 0.2 to 4 percent by weight. Suitable direct dyes to be used in the composition according to the invention are, e.g., triphenylmethane colorants, aromatic nitro colorants, azo colorants, quinone colorants or cationic or anionic colorants. Additional known and common colorants that are for colouring hair and that can be used in the colouring substance according to the invention are described in, among other places, E. Sagarin, “Cosmetics, Science and Technology”, Interscience Publishers Inc., New York (1957), pages 503 ff. and H. Janistyn, “Handbuch der Kosmetika und Riechstoffe”, Band 3 (1973), pages 388 ff. and K. Schrader “Grundlagen und Rezepturen der Kosmetika”, 2^{nd} edition (1989), pages 782-815.

Suitable hair-colouring pigments are practically insoluble colorants in the medium of application, and these pigments can be of organic or inorganic nature. Inorganic-organic mixed phase pigments are also possible. The pigments are preferably not nanopigments. The preferred particle size is from 1 to 200 μm, particularly 3 to 150 μm, particularly preferably 10 to 100 μm. Preferred are inorganic pigments.

The hair treatment composition according to the invention preferably additionally contains at least one hair-care substance in an amount of from 0.01 to 10, particularly preferably of from 0.05 to 5, percent by weight. Preferred hair-care substances are silicone compounds, as well as cationic substances which, because of groups that are cationic or that can be rendered cationic, particularly primary, secondary, tertiary or quaternary amine groups, have substantivity on human hair. Suitable cationic substances are selected from cationic tensides, betainic, amphoteric tensides, cationic polymers, silicone compounds with groups that are cationic or that can be rendered cationic, cationically derivatized proteins or protein hydrolysates and betaine.

Suitable silicone compounds are, for example, polydimethylsiloxane (INCI: dimethicone), α-hydro-ω-hydroxypolyoxydimethylsilylene (INCI: dimethiconol), cyclic dimethylpolysiloxane (INCI: cyclomethicone), trimethyl(octadecyloxy)silane (INCI: stearoxytrimethylsilan), dimethylsiloxane/glycol copolymer (INCI: (dimethicone copolyol), dimethylsiloxane/aminoalkyl siloxane copolymer having terminal hydroxyl groups (INCI: amodimethicone), monomethylpolysiloxane having lauryl side chains and terminal polyoxyethylene chains and/or terminal polyoxypropylene chains (INCI: lauryl methicone copolyol), dimethylsiloxane/glycol copolymer acetate (INCI: dimethicone copolyol acetate), dimethylsiloxane/aminoalkyl siloxane copolymer having terminal trimethylsilyl groups (INCI: trimethylsilylamodimethicone). Preferred silicone polymers are dimethicones, cyclomethicones and dimethiconols. Mixtures of silicone polymers are also suitable, for example a mixture of dimethicone and dimethiconol. The designations provided in brackets correspond to INCI (International Cosmetic Ingredients) nomenclature employed for the designation of cosmetic active ingredients and additives.

As a rule, additional known cosmetic additives can be added to the hair treatment composition according to the invention, for example, non-fixative non-ionic polymers, such as polyethylene glycols; non-fixative, anionic and natural polymers, as well as mixtures thereof, in amounts of preferably from 0.01 to 50 percent by weight. It is also possible to add perfume oils in an amount of from 0.01 to 5 percent by weight, opacifiers such as ethylene glycol distearate in an amount of from 0.01 to 5 percent by weight, wetting agents or emulsifiers from the classes of anionic, cationic, amphoteric or non-ionic tensides, such as fatty alcohol sulphates, ethoxylated fatty alcohols, fatty acid alcanolamides, such as esters of hydrated fatty acids from castor oil, in an amount of from 0.1 to 30 percent by weight, as well as moisturizers, dye-receptivity improving agents, light protection agents, anti-oxidative agents and preservatives in amounts of from 0.01 to 10 percent by weight.

A further object of the invention is a method for hair treatment, wherein

a composition, described above and according to the invention, is applied to the hair,

beforehand, simultaneously or subsequently, the hair is arranged into a defined shape under the application of heat and

subsequently the shape is fixed by cooling.

The shaping of the hair suitably occurs under heating to a temperature of at least T′ₜᵣₐₙₛ, and the shape of the hair is fixed by means of cooling to a temperature below T′ₜᵣₐₙₛ. Room temperature usually refers to the ambient temperature, preferably at least 20° C. and, in warmer climates, preferably at least 25° C. The application of the composition onto the hair can be carried out in different ways, for example, directly by spraying or indirectly by applying the composition first to the hand or a suitable device, for example a comb, a brush etc., followed by a subsequent distribution in or onto the hair. The consistency of the composition can, for example, be that of a solution, dispersion, lotion, thickened lotion, gel, foam or a semi-solid compound, creamy or waxy.

A further object of the invention is a method for impressing a second hairstyle on to a programmed, retrievable first hairstyle. In this connection, first a hairstyle (permanent shape) programmed by the method mentioned above is heated to a temperature above Tₜᵣₐₙₛ or between T′ₜᵣₐₙₛ and Tₜᵣₐₙₛ. The hair is then brought into the desired second (temporary) shape and the second shape is fixed by cooling to a temperature below Tₜᵣₐₙₛ.

A further object of the invention is a method for recovering a first hairstyle (permanent shape), programmed by means of the above-mentioned method. For this purpose, a hairstyle in a temporary shape, or a hairstyle deformed by cold forming, is heated to a temperature above Tₜᵣₐₙₛ. The permanent shape forms again, spontaneously and automatically.

The invention furthermore relates to a method for reprogramming a permanent hairstyle, previously programmed according to the above-mentioned method, into a different, new permanent shape. For this purpose, the original hairstyle is heated to a temperature above T′ₜᵣₐₙₛ and the hair is brought into a new shape. This new shape is subsequently fixed by means of cooling to a temperature below T′ₜᵣₐₙₛ.

The term “hairstyle” in the context of the invention is to be understood broadly and comprises, for example, also the degree of waviness or the degree of straightness of the hair. In the context of the invention, a programmed hairstyle is a group of hairs showing a specific shape due to shape memory polymers which have been cross-linked and fixed in a permanent shape. In the context of the invention, recovery of a programmed hairstyle means that the programmed hairstyle is recovered to a detectable degree after a deformation, preferably to a degree of at least 60%, particularly preferably to a degree of at least 80%, in relation to the shape that arises after a first relaxation cycle. The degree of recovery can, for example, be determined by means of measuring the length of a hair curl or hair strand. Cold-forming of a hairstyle means a change of the hairstyle at the ambient temperature, without the supply of additional heat by means of a hairdryer or similar devices. The deformation can, for example, be caused mechanically, for example, by means of simply hanging the curls under the influence of gravity, by means of combing or brushing the hair, as a result of the wind or humidity or as the result of mechanical influences during sleep or while lying down, etc.

Shape memory polymers in the context of the invention are polymers from which materials can be produced that have the property that they allow any shape (permanent shape) to be impressed upon them, to which shape they recover, spontaneously and without the application of external forces, after a deformation or after the impression of a second shape (temporary shape), simply as a result of heating or another energetic stimulus. At the same time, deformation and recovery are possible multiple times. The degree to which the original, permanent shape is achieved is, as a rule, somewhat less in a first relaxation cycle, consisting of deformation and recovery, than in subsequent cycles, presumably because of the elimination of faulty spots, textures, etc. still existing at the beginning. An especially high degree of recovery is then achieved, however, in the subsequent relaxation cycles. The degree of recovery in the first relaxation cycle is preferably at least 30%, more preferably at least 50%, and, in the subsequent relaxation cycles, preferably at least 60%, particularly preferably at least 80%. The degree may, however, also be 90% or more. The degree of recovery can be measured in accordance with usual curl retention measurements by simply determining the length of a treated hair curl or by means of known, suitable stress-strain experiments.

In the context of the invention, physically cross-linkable shape memory polymers are polymers with which the fixation of the impressed permanent shape due to cross-linking occurs by physical interactions. A cross-linking by means of physical interactions can be achieved when specific segments of the polymer chains assemble together into crystalline areas. The physical interactions can be charge transfer complexes, hydrogen bonds, dipolar interactions, hydrophobic interactions, Van der Waals interactions or ionic interactions of polyelectrolyte segments. The interactions can occur between different segments within one polymer strand (intramolecular) and/or between different polymer strands (intermolecular). The formation of the interactions can, for example, be initiated by means of cooling (in particular, in cases of crystallisation) and/or by means of drying, i.e., the removal of solvents.

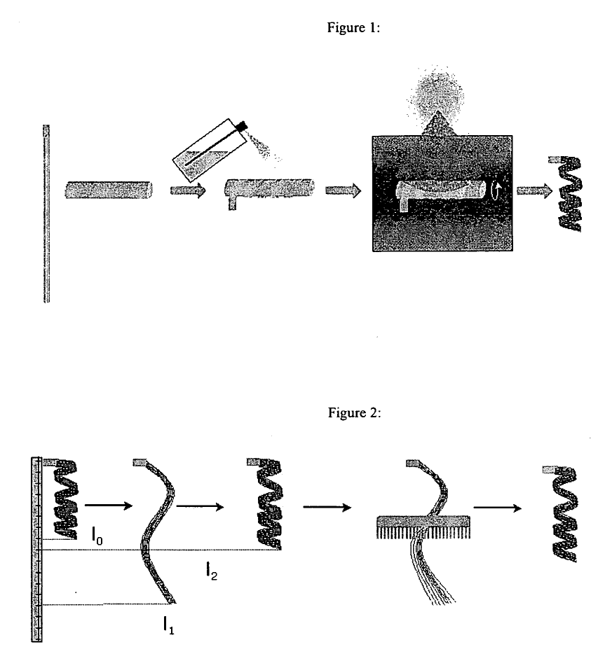

FIG. 1 schematically shows the method for treating hair. A strand of hair is wound onto a hair curler and sprayed with a liquid composition according to the invention, said composition containing a block polymer. The desired shape is fixed by means of heat treatment with a suitable energy source, for example, a hair dryer. Subsequently the curler is removed.

FIG. 2 shows the deformation of an originally prepared hairstyle and its recovery. The hair curl in the original (permanent) shape possesses a length l₀. The curl in the deformed shape has a length l₁. The curl in the recovered shape has a length l₂. The degree of recovery can be calculated as follows:
Recovery=(l₁−l₂)/(l₁−l₀).

As a measure for the evaluation of the shape memory properties of a composition, the memory factor can be employed, wherein this memory factor considers the formability of a permanent hairstyle into a temporary shape (shaping factor), as well as the restoration of the permanent shape, starting from the temporary shape (restoration factor, degree of recovery). If one starts with a straight strand of hair onto which a curly shape has been impressed as a permanent shape and onto which, subsequently, a second, straight shape has been impressed as a temporary shape, the shaping factor can be determined according to the following criteria:

| Degree of straightness | Shaping factor |
|---|---|
| Strongly wavy along the entire length | 0 |
| Slightly wavy from hairline to tip of hair | 1 |
| Hairline straight, tip with curl | 2 |
| Hairline straight, curvature at the tip | 3 |
| Straight all the way from the hairline to the tip | 4 |

The restoration factor can be determined according to the following criteria:

| Degree of restoration of the | |
|---|---|
| permanent shape | Restoration factor |
| 0% | 0 |
| 30% | 1 |
| 40% | 2 |
| 50% | 3 |
| 60% | 4 |
| 75% | 5 |
| 100% | 6 |

The memory factor M can be calculated with the following equation using the shaping factor f, the maximum shaping factor F=4, the particular restoration factor r and the maximum restoration factor R=6:
M=(f/F)*(r/R)*100
The memory factor should ideally be not below 20, preferably at least 25 or at least 30, particularly preferably between 35 and 100.

The following examples are intended to illustrate the object of the invention further.

### EXAMPLES

The production of the triblock copolymers used in the following examples is done using the respective diols that form the middle block as initiators and esterification with the respective polyesters that from the terminal blocks using customary esterification reactions.

Production of an Oligoester Diol

Dicarboxylic acid (1 mol) and diol (1.2 to 1.5 mol) are mixed at 100° C. by stirring. The esterification reaction is then started by adding p-toluenesulphonic acid (1-5 percent by weight, with relation to the total mass). After the reaction ends, the temperature is cooled to 40° C. and methylene chloride is added to dissolve the reaction mass. The product is precipitated in methanol, washed with methanol and dried in the vacuum-drying chamber. The average molar masses are determined by using GPC (calibration with polystyrene).

### Examples 1 to 56

Compositions, roughly 2-3 percent, of the following block copolymers are produced in ethanol/water (50:50) or ethanol/water/acetone (45:45:10).

| Middle | | | Example |
|---|---|---|---|
| block | Terminal blocks | Mn | no. |
| PEG-4k | PDL | 6,000 | 1 |
| PEG-6k | PDL | 8,000 | 2 |
| PEG-8k | PDL | 10,000 | 3 |
| PEG-8k | PDL | 12,000 | 4 |
| PEG-8k | P-LL-LA | 10,000 | 5 |
| PEG-8k | P-LL-LA | 9,000 | 6 |
| PEG-4k | P-DL-LA | 10,000 | 7 |
| PEG-6k | P-DL-LA | 10,000 | 8 |
| PEG-8k | P-DL-LA | 10,000 | 9 |
| PEG-6k | PCL | 8,000 | 10 |
| PEG-6k | PCL | 10,000 | 11 |
| PEG-8k | PCL | 10,000 | 12 |
| PEG-8k | PCL | 12,000 | 13 |
| PEG-8k | PCL | 15,000 | 14 |
| PEG-6k | P (CL-co-DL-LA) 30:10 | 10,000 | 15 |
| PEG-6k | P (CL-co-DL-LA) 20:20 | 10,000 | 16 |
| PEG-6k | P (CL-co-DL-LA) 10:30 | 10,000 | 17 |
| PEG-6k | P (CL-co-LL-LA) 30:10 | 10,000 | 18 |
| PEG-6k | P (CL-co-LL-LA) 20:20 | 10,000 | 19 |
| PEG-6k | P (CL-co-LL-LA) 10:30 | 10,000 | 20 |
| PEG-6k | P(CL-co-PDL) 30:10 | 10,000 | 21 |
| PEG-6k | P(CL-co-PDL) 20:20 | 10,000 | 22 |
| PEG-6k | P(CL-co-PDL) 10:30 | 10,000 | 23 |
| PEG-6k | P (LL-LA-co-PDL) 30:10 | 10,000 | 24 |
| PEG-6k | P (LL-LA-co-PDL) 20:20 | 10,000 | 25 |
| PEG-6k | P (LL-LA-co-PDL) 10:30 | 10,000 | 26 |
| PEG-6k | PCL-b-P-DL-LA | 10,000 | 27 |
| PEG-4k | PDL | 6,000 | 1 |
| PEG-6k | PCL-b-P-DL-LA | 12,000 | 28 |
| PEG-6k | PCL-b-P-DL-LA | 15,000 | 29 |
| PEG-8k | PCL-b-P-DL-LA | 10,000 | 30 |
| PEG-8k | PCL-b-P-DL-LA | 12,000 | 31 |
| PEG-8k | PCL-b-P-DL-LA | 15,000 | 32 |
| PEG-6k | PCL-b-P-LL-LA | 10,000 | 33 |
| PEG-6k | PCL-b-P-LL-LA | 12,000 | 34 |
| PEG-6k | PCL-b-P-LL-LA | 15,000 | 35 |
| PEG-8k | PCL-b-P-LL-LA | 10,000 | 36 |
| PEG-8k | PCL-b-P-LL-LA | 12,000 | 37 |
| PEG-8k | PCL-b-P-LL-LA | 15,000 | 38 |
| PEG-6k | PCL-b-PPDL | 10,000 | 39 |
| PEG-6k | PCL-b-PPDL | 12,000 | 40 |
| PEG-6k | PCL-b-PDL | 15,000 | 41 |
| PEG-8k | PCL-b-PPDL | 10,000 | 42 |
| PEG-8k | PCL-b-PPDL | 12,000 | 43 |
| PEG-8k | PCL-b-PDL | 15,000 | 44 |
| PEG-6k | P-LL-LA-b-PPDL | 10,000 | 45 |
| PEG-6k | P-LL-LA-b-PPDL | 12,000 | 46 |
| PEG-6k | P-LL-LA-b-PPDL | 15,000 | 47 |
| PEG-8k | P-LL-LA-b-PPDL | 10,000 | 48 |
| PEG-8k | P-LL-LA-b-PPDL | 12,000 | 49 |
| PEG-8k | P-LL-LA-b-PPDL | 15,000 | 50 |
| PEG-6k | P-DL-LA-b-PPDL | 10,000 | 51 |
| PEG-6k | P-DL-LA-b-PPDL | 12,000 | 52 |
| PEG-6k | P-DL-LA-b-PPDL | 15,000 | 53 |
| PEG-4k | PDL | 6,000 | 1 |
| PEG-8k | P-DL-LA-b-PPDL | 10,000 | 54 |
| PEG-8k | P-DL-LA-b-PPDL | 12,000 | 55 |
| PEG-8k | P-DL-LA-b-PPDL | 15,000 | 56 |

### Examples 57 to 100

Compositions, roughly 2-3 percent, of the following block copolymers are produced in ethanol/water/acetone (25:25:50).

| Middle | | | Example |
|---|---|---|---|
| block | Terminal blocks | Mn | no. |
| Dimerdiol | PPDL | 5,000 | 57 |
| Dimerdiol | PPDL | 10,000 | 58 |
| Dimerdiol | P-LL-LA | 5,000 | 59 |
| Dimerdiol | P-LL-LA | 10,000 | 60 |
| Dimerdiol | P-DL-LA | 5,000 | 61 |
| Dimerdiol | P-DL-LA | 10,000 | 62 |
| Dimerdiol | PCL | 5,000 | 63 |
| Dimerdiol | PCL | 10,000 | 64 |
| D-OEt 1000 | PPDL | 5,000 | 65 |
| D-OEt 1000 | PPDL | 10,000 | 66 |
| D-OEt 1000 | P-LL-LA | 5,000 | 67 |
| D-OEt 1000 | P-LL-LA | 10,000 | 68 |
| D-OEt 1000 | P-DL-LA | 5,000 | 69 |
| D-OEt 1000 | P-DL-LA | 10,000 | 70 |
| D-OEt 1000 | PCL | 5,000 | 71 |
| D-OEt 1000 | PCL | 10,000 | 72 |
| D-OEt 2000 | PPDL | 5,000 | 73 |
| D-OEt 2000 | PPDL | 10,000 | 74 |
| D-OEt 2000 | P-LL-LA | 5,000 | 75 |
| D-OEt 2000 | P-LL-LA | 10,000 | 76 |
| D-OEt 2000 | P-DL-LA | 5,000 | 77 |
| D-OEt 2000 | P-DL-LA | 10,000 | 78 |
| D-OEt 2000 | PCL | 5,000 | 79 |
| D-OEt 2000 | PCL | 10,000 | 80 |
| D-OEs 1000 | PPDL | 10,000 | 81 |
| D-OEs 1000 | PCL | 10,000 | 82 |
| D-OEs 1000 | P-LL-LA | 10,000 | 83 |
| D-OEs 1000 | P-DL-LA | 10,000 | 84 |
| D-OEs 2000 | PPDL | 10,000 | 85 |
| D-OEs 2000 | PCL | 10,000 | 86 |
| D-OEs 2000 | P-LL-LA | 10,000 | 87 |
| D-OEs 2000 | P-DL-LA | 10,000 | 88 |
| D-OEs 3000 | PPDL | 10,000 | 89 |
| D-OEs 3000 | PCL | 10,000 | 90 |
| D-OEs 3000 | P-LL-LA | 10,000 | 91 |
| D-OEs 3000 | P-DL-LA | 10,000 | 92 |
| D-OEs 4000 | PPDL | 10,000 | 93 |
| D-OEs 4000 | PCL | 10,000 | 94 |
| D-OEs 4000 | P-LL-LA | 10,000 | 95 |
| D-OEs 4000 | P-DL-LA | 10,000 | 96 |
| D-OEs 5000 | PPDL | 10,000 | 97 |
| D-OEs 5000 | PCL | 10,000 | 98 |
| D-OEs 5000 | P-LL-LA | 10,000 | 99 |
| D-OEs 5000 | P-DL-LA | 10,000 | 100 |

Examples: Hair Treatment with Triblock Copolymers

| | Example 1 | Example 2 | Example 3 | Example 4 | Example 5 |
|---|---|---|---|---|---|
| Block | 3 percent | | | | |
| copolymer 1 | by weight | | | | |
| Block | | 3 percent | | | |
| copolymer 5 | | by weight | | | |
| Block | | | 3 percent | | |
| copolymer 6 | | | by weight | | |
| Block | | | | 3 percent | |
| copolymer | | | | by weight | |
| 10 | | | | | |
| Block | | | | | 3 percent |
| copolymer | | | | | by weight |
| 16 | | | | | |
| Treatment | Heat | Heat | Heat | Heat | Heat |
| Solvent | Et/W | Et/W | Et/W | Et/W/Ac | Et/W |
| | 50/50 | 50/50 | 50/50 | 45/45/10 | 50/50 |
| Tₜᵣₐₙₛ, | 57, 85 | 40, 57 | 40, 56 | 44, 53 | 50-60 |
| T′ₜᵣₐₙₛ | | | | | |
| [° C.] | | | | | |
| Memory | 25 | 42 | 40 | 30 | 40 |
| factor M | | | | | |

Ac = acetone,
W = water,
Et = ethanol

Hair Coating and Production of the Permanent Shape:

A strand of hair having a length of 20 cm, having been moistened with water, is wound onto a curler and the polymer solution is applied thereon (20 to 30 mg polymer/g hair). The treated strand of hair is then heated to 70° C. for 30 minutes. After cooling to room temperature (about 25° C.), the hair curler is removed. The curled strand (impressed permanent shape) had a length of about 4.5 cm.

Production of the Temporary Shape and Recovery of the Permanent Shape:

In order to produce a temporary shape (e.g., straight), the curled strand is heated to about 55° C., extended to the full original length (20 cm) and cooled again to room temperature. The permanent shape can be recovered by heating the straight strand to about 55° C. At this temperature, the strand spontaneously retracted to the permanent (curly) shape.

In order to produce a temporary shape (e.g., straight) again, the curled strand is again heated to 55° C., extended to its full length (20 cm) and cooled to room temperature.

Examples of a Cosmetic Hair Composition

### Example 1

### Aerosol Hair Blower Lotion

| | | |
|---|---|---|
| 3.335 | g | Block polymer no. 5 |
| 0.20 | g | Perfume |
| 0.02 | g | Baysilon ® Oil PD 5 (phenyl trimethicone) |
| 10.00 | g | Water |
| Ad 100 | g | Ethanol |

The active ingredient solution is filled into an aerosol can with DME as a propellant, in the ratio 45:55.

### Example 2

### Aerosol Hair Blower Lotion

| | | |
|---|---|---|
| 3.3 | g | Block polymer no. 1 |
| 3.5 | g | Octylacrylamide/acrylic acid/butylaminoethylmethacrylate/ |
| | | methylmethacrylate/hydroxypropyl methacrylate copolymer |
| | | (Arnphomer ® LV 71) |
| 0.57 | g | Aminomethyl propanol 95% |
| 0.20 | g | Perfume |
| 0.02 | g | Baysilon ® Oil PD 5 (phenyl trimethicone) |
| 10.00 | g | Water |
| Ad 100 | g | Ethanol |

The active ingredient solution is filled into an aerosol can with DME as a propellant, in the ratio 45:55.

### Example 3

### Aerosol Hair Blower Lotion

| | | |
|---|---|---|
| 3.335 | g | Block polymer no. 10 |
| 3.335 | g | Vinyl acetate/crotonic acid copolymer (Luviset ® CA 66) |
| 0.378 | g | Aminomethyl propanol 95% |
| 0.20 | g | Perfume |
| 0.02 | g | Baysilon ® oil PD 5 (phenyl trimethicone) |
| Ad 100 | g | Ethanol |

The active ingredient solution is filled into an aerosol can with propane/butane (2.7 bar) as a propellant, in the ratio 65:35.

### Example 4

### Hairspray

| | | |
|---|---|---|
| 2.5 | g | Block polymer no. 9 |
| 3.3 | g | T-butyl acrylate/ethyl acrylate/methacrylic acid copolymer |
| | | (Luvimer ® |
| | | 100 P) |
| 0.844 | g | Aminomethyl propanol 95% |
| 0.20 | g | Perfume |
| 0.02 | g | Baysilon ® oil PD 5 (phenyl trimethicone) |
| 10.00 | g | Water |
| Ad 100 | g | Ethanol |

The active ingredient solution was filled into an aerosol can with DME as a propellant, in the ratio 45:55.

### Example 5

### Aerosol Fixing Foam

| | | |
|---|---|---|
| 6.1 | g | Block polymer no. 7 |
| 11.9 | g | Ethanol |
| 0.14 | g | Celquat ® L200 (copolymer of hydroxyethylcellulose and |
| | | diallyl dimethyl ammonium chloride; polyquaternium-4) |
| 0.2 | g | Laureth-4 |
| 0.2 | g | Panthenol |
| 0.2 | g | Perfume |
| 0.07 | g | Cetyl trimethyl ammonium bromide |
| 4 | g | Propane |
| 4 | g | Butane |
| Ad 100 | g | Water |

### Example 6

### Aerosol Fixing Foam

| | | |
|---|---|---|
| 6.1 | g | Block polymer no. 6 |
| 11.9 | g | Ethanol |
| 0.3 | g | Vinylpyrrolidone/dimethylamino ethyl methacrylate |
| | | copolymer (Gafquat ® 755 N) |
| 0.2 | g | Laureth-4 |
| 0.2 | g | Panthenol |
| 0.2 | g | Perfume |
| 0.07 | g | Cetyl trimethyl ammonium bromide |
| 4 | g | Propane |
| 4 | g | Butane |
| Ad 100 | g | Water |

### Example 7

### Aerosol Fixing Foam

| | | |
|---|---|---|
| 4.1 | g | Block polymer no. 8 |
| 0.6 | g | PVP/Vinyl caprolactam/DMAPA acrylates copolymer |
| | | (Aquaflex ® SF 40) |
| 0.07 | g | Aminomethyl propanol 95% |
| 18.9 | g | Ethanol |
| 0.4 | g | PEG 25 PABA |
| 0.2 | g | Laureth-4 |
| 0.2 | g | Panthenol |
| 0.2 | g | Perfume |
| 0.07 | g | Cetyl trimethyl ammonium chloride |
| 4 | g | Propane |
| 4 | g | Butane |
| Ad 100 | g | Water |

### Example 8

### Aerosol Fixing Foam

| | | |
|---|---|---|
| 5.5 | g | Block polymer no. 4 |
| 8.9 | g | Ethanol |
| 0.45 | g | Polyvinylpyrrolidone (PVP K 90) |
| 0.4 | g | PEG 25 PABA |
| 1.0 | g | Abilquat ® 3272 |
| | | (Quaternium-80, 50% in propylene glycol) |
| 0.15 | g | Betaine |
| 0.2 | g | Perfume |
| 0.07 | g | Cetyl trimethyl ammonium chloride |
| 4 | g | Propane |
| 4 | g | Butane |
| Ad 100 | g | Water |

### Example 9

### Aerosol Fixing Foam

| | | |
|---|---|---|
| 3.5 | g | Block polymer no. 6 |
| 8.9 | g | Ethanol |
| 0.45 | g | Polyvinylpyrrolidone (PVP K 90) |
| 0.4 | g | PEG 25 PABA |
| 1.0 | g | Abilquat ® 3272 |
| | | (Quaternium-80, 50% in propylene glycol) |
| 0.2 | g | Perfume |
| 0.07 | g | Cetyl trimethyl ammonium chloride |
| 4 | g | Propane |
| 4 | g | Butane |
| Ad 100 | g | Water |

### Example 10

### Aerosol Fixing Foam

| | | |
|---|---|---|
| 5.5 | g | Block polymer no. 5 |
| 8.9 | g | Ethanol |
| 0.45 | g | Polyvinylpyrrolidone (PVP K 30) |
| 0.4 | g | PEG 25 PABA |
| 0.3 | g | Celquat ® L200 (copolymer of hydroxyethylcellulose and |
| | | diallyl dimethyl ammonium chloride; polyquaternium-4) |
| 0.2 | g | Perfume |
| 0.07 | g | Cetyl trimethyl ammonium bromide |
| 4 | g | Propane |
| 4 | g | Butane |
| Ad 100 | g | Water |

### Example 11

### Non-Aerosol Hair Blower Lotion

| | | |
|---|---|---|
| 4.5 | g | Block polymer no. 9 |
| 0.45 | g | Vinyl acetate/crotonic acid/polyethylene oxide copolymer |
| | | (Aristoflex ® A) |
| 27 | g | Ethanol |
| 0.7 | g | PEG 25 PABA |
| 0.35 | g | Panthenol |
| 0.1 | g | Betaine |
| 0.25 | g | Perfume |
| 0.21 | g | PEG 40 hydrogenated castor oil |
| 0.20 | g | Cetyl trimethyl ammonium chloride |
| Ad 100 | g | Water |

The composition can be filled into a package with a pump spray device and used as a non-aerosol spray.

### Example 12

### Non-Aerosol Hair Blower Lotion

| | | |
|---|---|---|
| 4.5 | g | Block polymer no. 6 |
| 20 | g | Ethanol |
| 0.7 | g | PEG 25 PABA |
| 0.35 | g | Panthenol |
| 0.1 | g | Betaine |
| 0.25 | g | Perfume |
| 0.21 | g | PEG 40 hydrogenated castor oil |
| 0.20 | g | Cetyl trimethyl ammonium bromide |
| Ad 100 | g | Water |

The composition can be filled into a package with a pump spray device and used as a non-aerosol spray.

### Example 13

### Non-Aerosol Hair Blower Lotion

| | | |
|---|---|---|
| 4.5 | g | Block polymer no. 9 |
| 0.2 | g | Polyquaternium-46 (Luviquat Hold) |
| 27 | g | Ethanol |
| 0.7 | g | PEG 25 PABA |
| 0.35 | g | Panthenol |
| 0.1 | g | Betaine |
| 0.25 | g | Perfume |
| 0.21 | g | PEG 40 hydrogenated castor oil |
| 0.20 | g | Cetyl trimethyl ammonium chloride |
| Ad 100 | g | Water |

The composition can be filled into a package with a pump spray device and used as a non-aerosol spray.

### Example 14

### Non-Aerosol Hair Blower Lotion

| | | |
|---|---|---|
| 3.1 | g | Block polymer no. 13 |
| 0.05 | g | Celquat ® L200 (copolymer of |
| | | hydroxyethylcellulose and diallyl dimethyl ammonium |
| | | chloride; polyquaternium-4) |
| 0.5 | g | Polyvinylpyrrolidone/vinyl acetate copolymer (VA 64) |
| 27 | g | Ethanol |
| 0.7 | g | PEG 25 PABA |
| 0.35 | g | Panthenol |
| 0.1 | g | Betaine |
| 0.25 | g | Perfume |
| 0.21 | g | PEG 40 hydrogenated castor oil |
| 0.20 | g | Cetyl trimethyl ammonium bromide |
| Ad 100 | g | Water |

The composition can be filled into a package with a pump spray device and used as a non-aerosol spray.

### Example 15

### Non-Aerosol Hair Blower Lotion

| | | |
|---|---|---|
| 4.1 | g | Block polymer no. 6 |
| 1.0 | g | Block polymer no. 9 |
| 0.15 | g | Polyquaternium-46 (Luviquat Hold) |
| 27 | g | Ethanol |
| 0.7 | g | PEG 25 PABA |
| 0.35 | g | Panthenol |
| 0.25 | g | Perfume |
| 0.21 | g | PEG 40 hydrogenated castor oil |
| 0.20 | g | Cetyl trimethyl ammonium chloride |
| Ad 100 | g | Water |

The composition can be filled into a package with a pump spray device and used as a non-aerosol spray.

### Example 16

### Non-Aerosol Hair Blower Lotion

| | | |
|---|---|---|
| 6.0 | g | Block polymer no. 6 |
| 2.0 | g | Vinyl caprolactam/dimethylamino ethyl methacrylate |
| | | copolymer (Advantage ® S) |
| 28.5 | g | Ethanol |
| 0.25 | g | Perfume |
| 0.20 | g | Cetyl trimethyl ammonium chloride |
| Ad 100 | g | Water |

The composition can be filled into a package with a pump spray device and used as a non-aerosol spray.

### Example 17

### Spray Fixer

| | | |
|---|---|---|
| 6.0 | g | Block polymer no. 6 |
| 2.0 | g | Octylacrylamide/acrylates/butylaminoethylmethacrylate |
| | | copolymer (Amphomer ®) |
| 28.5 | g | Ethanol |
| 0.6 | g | Aminomethyl propanol 95% |
| 0.25 | g | Perfume |
| 0.20 | g | Cetyl trimethyl ammonium bromide |
| Ad 100 | g | Water |

The composition can be filled into a package with a pump spray device and used as a non-aerosol spray.

### Example 18

### Spray Fixer

| | | |
|---|---|---|
| 3.0 | g | Block polymer no. 6 |
| 0.65 | g | Octylacrylamide/acrylates/butylaminoethylmethacrylate |
| | | copolymer (Amphomer ®) |
| 0.2 | g | Celquat ® L200 (copolymer |
| | | of hydroxyethylcellulose and diallyl |
| | | dimethyl ammonium chloride; polyquaternium-4) |
| 28.5 | g | Ethanol |
| 0.6 | g | Aminomethyl propanol 95% |
| 0.25 | g | Perfume |
| 0.20 | g | Cetyl trimethyl ammonium chloride |
| Ad 100 | g | Water |

The composition can be filled into a package with a pump spray device and used as a non-aerosol spray.

### Example 19

### Spray Gel

| | | |
|---|---|---|
| 5.2 | g | Block polymer no. 7 |
| 20 | g | Ethanol |
| 0.1 | g | Aminomethyl propanol 95% |
| 0.2 | g | PEG 40 hydrogenated castor oil |
| 0.2 | g | Perfume |
| 1.5 | g | Carbomer |
| Ad 100 | g | Water |

The composition can be filled into a package with a pump spray device and used as a non-aerosol spray.

### Example 20

### Spray gel

| | | |
|---|---|---|
| 4.0 | g | Block polymer no. 6 |
| 3.0 | g | VP/VA copolymer (Luviskol VA 64) |
| 18 | g | Ethanol |
| 0.1 | g | Aminomethyl propanol 95% |
| 0.2 | g | PEG 40 hydrogenated castor oil |
| 0.2 | g | Perfume |
| 1.5 | g | Carbomer |
| Ad 100 | g | Water |

The composition can be filled into a package with a pump spray device and used as a non-aerosol spray.

### Example 21

### Pump Fixing Foam

| | | |
|---|---|---|
| 5.5 | g | Block polymer no. 6 |
| 8.9 | g | Ethanol |
| 0.2 | g | Cocamidopropyl hydroxysultaine |
| 0.2 | g | Cetyl trimethyl ammonium chloride |
| 0.15 | g | Perfume |
| 0.1 | g | Citric acid |
| 0.1 | g | Betaine |
| Ad 100 | g | Water |

### Example 22

### Pump Fixing Foam

| | | |
|---|---|---|
| 4.0 | g | Block polymer no. 9 |
| 0.3 | g | Celquat ® L200 (copolymer of hydroxyethylcellulose |
| | | and diallyl dimethyl ammonium chloride; |
| | | polyquaternium-4) |
| 8.9 | g | Ethanol |
| 0.2 | g | Cocamidopropyl hydroxysultaine |
| 0.2 | g | Cetyl trimethyl ammonium chloride |
| 0.15 | g | Perfume |
| 0.1 | g | Citric acid |
| 0.1 | g | Betaine |
| Ad 100 | g | Water |

### Example 23

### Pump Fixing Foam

| | | |
|---|---|---|
| 4.4 | g | Block polymer no. 8 |
| 2.4 | g | Vinyl caprolactam/VP/dimethylamino ethyl methacrylate |
| | | copolymer (Advantage ® LC-E) |
| 8.9 | g | Ethanol |
| 0.4 | g | Cocamidopropyl hydroxysultaine |
| 0.15 | g | Perfume |
| 0.1 | g | Citric acid |
| Ad 100 | g | Water |

### Example 24

### Pump Fixing Foam

| | | |
|---|---|---|
| 5.1 | g | Block polymer no. 14 |
| 0.35 | g | T-butyl acrylate/ethyl acrylate/methacrylic acid copolymer |
| | | (Luvimer ® 30 E) |
| 0.05 | g | Chitosan (C XII) |
| 0.89 | g | Aquaflex ® SF 40 (PVP/vinyl caprolactam/DMAPA |
| | | acrylates copolymer) |
| 0.06 | g | Aminomethyl propanol 95% |
| 8.9 | g | Ethanol |
| 0.4 | g | Cocamidopropyl hydroxysultaine |
| 0.15 | g | Perfume |
| 0.1 | g | Citric acid |
| Ad 100 | g | Water |

In each of the previously mentioned cosmetic hair compositions, another of the block polymers no. 1 to 100 can be used alternatively or in addition.

The abbreviations used in the examples have the following meanings:
PEG-4k, PEG-6k, PEG-8k: Polyethylene glycol with a molecular weight of 4,000, 6,000 or 8,000, respectively
PDL: Pentadecalactone; lactone of 15-hydroxypentadecanoic acid
PPDL: Poly(pentadecalactone), poly(15-hydroxypentadecanoic acid)
P-LL-LA: Poly(L-lactic acid)
P-DL-LA: Poly(DL-lactic acid)
PCL: Poly(ε-caprolactone)
P (CL-co-LA): Poly (e-caprolactone-co-DL-lactic acid)
PCL-b-PPDL: Poly(e-caprolactone)-block-poly(pentadecalactone)
PEG(4k)-DMA, PEG(8k)-DMA, PEG(10k)-DMA:
Poly(ethylene glycol)-dimethacrylate
PLGA(7k)-DMA: Poly(L-lactide-co-glycolide)-dimethacrylate
PCl (10k)-DMA: Poly(ε-caprolactone)-dimethacrylate
D-OEt 1000: Dimerdiol-oligoether Mn=1000 (Sovermol 909)
D-OEt 2000: Dimerdiol-oligoether Mn=2000 (Sovermol 910)
D-OEs 1000: Oligoester-diol Mn=1000

from glycerol monostearate and azelaic acid
D-OEs 2000: Oligoester-diol Mn=2000

from hydroxystearyl alcohol and adipic acid
D-OEs 3000: Oligoester-diol Mn=3000

from hydroxystearyl alcohol and azelaic acid
D-OEs 4000: Oligoester-diol Mn=4000

from hydroxystearyl alcohol and azelaic acid
D-OEs 5000: Oligoester-diol Mn=5000

from hydroxystearyl alcohol and azelaic acid

1. A cosmetic composition comprising, in a suitable cosmetic foundation, at least one block polymer having at least a first block which is a polyol selected from polyethers, oligoethers, hydrocarbons having a molecular weight of at least 400 g/mol and at least two alcoholic hydroxyl groups, oligoester diols and polyesters of dicarboxylic acids with diols, and at least two additional blocks which are polyesters of hydroxycarboxylic acids or their lactones.

2. The composition according to claim 1, wherein the block polymer
a) has at least one hard segment with a first transition temperature T′ₜᵣₐₙₛ which lies above room temperature, and
b) has at least one soft segment with a second transition temperature Tₜᵣₐₙₛ which lies below T′ₜᵣₐₙₛ.

3. The composition according to claim 1, wherein the block polymer has the formula A(B)ₙ wherein A is derived from an n-valent polyether or oligoether, from hydrocarbons having a molecular weight of at least 400 g/mol and n alcoholic hydroxyl groups, from oligoester diols or from a polyester of a dicarboxylic acid with one diol, B represents a poly(hydroxycarboxylic acid) block, and n represents a number larger than or equal to two.

4. The composition according to claim 3, wherein
A is selected from polyalkylene glycol ethers of polyvalent alcohols, poly(tetrahydrofurane), dimerdiol, dimerdiol oligoethers, oligoester diols,
B is selected from poly(ε-caprolactone), poly(pentadecanolactone), polylactides, polyglycolides, poly(lactide-co glycolide), and
n is 2, 3 or 4.

5. The composition according to claim 3, wherein the block polymer has a single polyol block as the middle block, two terminal polyester blocks of hydroxycarboxylic acids or lactones thereof with terminal alcoholic hydroxyl groups.

6. The composition according to claim 5, wherein the block polymer has the general formula
HO—[B1-C(═O)O-]n₁[Y—O—]ₙ₂[C(═O)—B2-O—]ₙ₃H
wherein B1 and B2 are the same or different and represent branched, cyclic or linear alkylene groups with 1 to 40 C atoms, Y represents a branched, cyclic or linear alkylene group with 2 to 30 C atoms or a polyester block of dicarboxylic acid and diol, and n1, n2 and n3 are the same or different numbers larger than zero.

7. The composition according to claim 6, wherein B1 and B2 represent branched, cyclic or linear alkylene groups with 2 to 20 C atoms, and Y represents ethylene groups and/or propylene groups.

8. The composition according to claim 7, wherein B1 and B2 represent branched or linear alkylene groups with 2 to 20 C atoms, Y represents an ethylene group, and n1, n2 and n3 are selected such that the molecular weight of the block polymer is greater than or equal to 2,000.

9. The composition according to claim 1, wherein the block polymer has a degree of crystallinity ranging from 3 to 80% and wherein the ratio of the moduli of elasticity below and above Tₜᵣₐₙₛ is at least 20.

10. The composition according to claim 1, wherein the block polymer is present in an amount ranging of from 0.01 to 25 percent by weight.

11. The composition according to claim 1, wherein at least one additional active ingredient is present in the amount ranging from 0.01 to 25 percent by weight, and wherein the active ingredient is selected from hair-care substances, hair-fixing substances and hair-coloring substances.

12. A cosmetic substance comprising a composition according to claim 1 in the form of a lotion, a spray lotion, a cream, a gel, a foam-gel, an aerosol spray, a non-aerosol spray, an aerosol foam, a non-aerosol foam, an o/w or w/o emulsion, a micro-emulsion or a hair wax.

13. (canceled)

14. A method for hair treatment, comprising
applying to the hair a composition according to claim 1,
arranging the hair into a defined shape under the application of heat before, simultaneously or subsequently the step of applying the composition, and
subsequently fixing the shape of the hair by cooling.

15. The method according to claim 14, wherein the hair is shaped by heating to a temperature of at least T′ₜᵣₐₙₛ, and subsequent fixing the shape of the hair is achieved by cooling to a temperature below T′ₜᵣₐₙₛ.

16. The method according to claim 15, wherein shaping of the hair takes place under heating
to a temperature between T′ₜᵣₐₙₛ and Tₜᵣₐₙₛ,
the hair is brought into a second shape, and
the second shape is fixed by means of cooling to a temperature below Tₜᵣₐₙₛ.

17. A method for the recovery of a previously programmed hairstyle by heating to a temperature above Tₜᵣₐₙₛ a hairstyle in a temporary shape according to claim 15 or a hairstyle deformed by means of cold forming.

18. A method for converting a hairstyle previously created by a method according to claim 14 into a new hairstyle, wherein
the hairstyle is heated to a temperature above T′ₜᵣₐₙₛ
the hairstyle is brought into a new shape and
the new shape is subsequently fixed by means of cooling to a temperature below T′ₜᵣₐₙₛ.
